(19) United States
(12) Patent Application Publication
(10) Pub. No.: US 2015/0157211 A1
(43) Pub. Date: Jun. 11, 2015

(54) Vascular Access Detection Device and Method

(71) Applicant: SenorMed, Inc., Knoxville, TN (US)

(72) Inventor: Michael C. Doody, Knoxville, TN (US)

(21) Appl. No.: 14/099,164

(22) Filed: Dec. 6, 2013

(51) Int. Cl. A61B 5/00; A61B 17/34; A61M 5/46; A61B 5/06; A61B 5/153

(52) U.S. Cl. CPC A61B 5/0086; A61B 5/065; A61B 5/489; A61B 5/1535; A61M 5/46; A61B 17/3468; A61B 17/3478; A61B 2010/045

(57) ABSTRACT

A vascular access detection device and method for ensuring the proper insertion of a needle into a target is disclosed. A light source generates a beam of light which is directed along the needle's longitudinal axis so that said beam of light passes though the hollow shaft of the needle. The needle may be guided to a target inside a patient's body. Reflection and scattering of light by portions of the patient's body may be monitored to assist in determining when the target inside of the patient's body has been pierced by the needle.

### CROSS-REFERENCE TO RELATED APPLICATIONS

Not Applicable

### STATEMENT REGARDING FEDERALLY-SPONSORED RESEARCH OR DEVELOPMENT

Not Applicable

### BACKGROUND OF THE INVENTION

1. Field of Invention

The invention relates to medical devices and procedures, and in particular, to apparatus and methods used to gather biological samples and introduce medical devices and therapeutic agents.

2. Description of the Related Art

The use of needles, trocars, or cannulae in the medical and veterinary professions, for example to draw blood or introduce fluids or medical agents into the body of a person or animal (hereinafter “patient”), is known. The proper insertion and guidance of a needle to a target location in a patient's body is challenging and relies on an imprecise combination of a clinician's sense of vision, sense of touch, and intuition. The process of guiding the needle requires time, skill, and concentration and, if done incorrectly, can be painful for the patient or lead to medical complications, such as for example collapsed veins or improper delivery of therapeutic agents and medical devices.

Clinicians currently rely on training to ensure the proper insertion of needles during medical procedures and develop skills concerning how to interpolate the location of the needle's tip in relation to an intended target location in the patient's body through vision, touch, and intuition. Many countries require clinicians who draw blood, known as phlebotomists, to be certified by associations like the American Society of Phlebotomy Technicians to verify that they have the necessary training and experience to properly insert and guide needles for medical treatment. Additionally, clinicians often rely on superficial blood vessels, which may be suboptimal or not be well-presented, and tourniquets, which can make superficial blood vessels more visible and reduce the risk of collapsing a vein, but at the expense of patient discomfort and additional time.

In accordance with the difficulties in the present art, a device or method that can provide clinicians greater certainty of a needle's location within a patient's body and reduce the risk and discomfort for patients is desired.

### BRIEF SUMMARY OF THE INVENTION

The present invention is a vascular access and detection device and method for assisting clinicians to perform phlebotomies and related procedures by using a light source, and preferably a laser light source, to shine a beam of light through the hollow shaft of a needle. When the needle's tip pierces the skin, light reflects and scatters off of, and back through, underlying tissue to illuminate the area where the needle's tip is located within the tissue, which the clinician uses to visually determine the depth and relative location of the tip to internal structures. If the tip pierces a structure containing opaque or semi-transparent fluid, such as for example a blood vessel, the tissue and fluids in the structure may at least partially quench the beam of light. The beam of light may thus be attenuated and reflective illumination may cease. When the needle is in place, biological samples can be collected or medical devices and agents can be internally introduced to the target location, for example inside a vein or artery of the patient.

The light source used in the vascular access and detection device and method invention described herein is adapted for indicating to a clinician the location of the tip of a needle when such needle is inserted into a patient's body. The hollow body of the needle can be used to direct light to the tip of the needle, as by directing a laser light through the needle to the tip. It will be understood that such laser light should have an intensity appropriate for illuminating the tissue surrounding the tip of the needle and not for therapeutic uses such as ablating or cauterizing the tissue and surrounding fluids. The colors and wavelength of the light generated by the light source can also be adapted to more readily be reflected off of and scattered, or absorbed by, tissue, to be transmitted through skin, and to be attenuated by targeted structures or the fluids within, such as blood vessels and blood.

It is to be noted that, as a medical device, special care must be taken to keep the vascular access and detection device sterile. To ensure the device is free of contamination and does not become contaminated when used on a patient, in several embodiments, the light source does not directly interface with the needle. Instead, in several embodiments, an interface bridge or adaptor may be used between the needle and the light source. The interface bridge allows the light to pass though to the needle and maintains a physical barrier between the needle and the light source. The interface bridge may be disposable or reusable. The interface bridge may also include other safety features, or engage safety features of the light source, that prevent the light source from interfacing directly with needles or generating a beam of light unless an interface bridge is in place.

### BRIEF DESCRIPTION OF THE DRAWINGS

The above-mentioned features of the invention will become more clearly understood from the following detailed description of the invention read together with the drawings in which:

FIG. 1 is a diagram showing the attenuation spectrum of human skin;

FIG. 2 is an exploded perspective view of one embodiment of the vascular access detection device constructed in accordance with several features of the present general inventive concept;

FIG. 3 is a perspective view of one embodiment of a light source;

FIG. 4 is a perspective view of one embodiment of an interface bridge;

FIG. 5 is a flowchart of one embodiment of a vascular access detection method according to several features of the present general inventive concept; and

FIGS. 6A-6C are a series of perspective views of a patient's arm demonstrating examples of reflective patterns useful in interpreting where the tip of a needle is when using one embodiment of the vascular access detection device.

### DETAILED DESCRIPTION OF THE INVENTION

With reference to the accompanying figures, the present invention is a vascular access and detection device 01 and method for assisting clinicians to perform phlebotomies and related procedures by using a light source 10 to shine a beam of light through the hollow shaft of a needle 30. When the needle's tip 32 pierces the skin 42 of a patient, light reflects and scatters off of underlying tissue to illuminate the area proximate to the tip's location within the tissue, which a clinician may detect and use, for example, to determine the depth and relative location of the tip 32 to internal structures 41 of the patient. If the tip 32 pierces a structure 41 containing opaque or semi-transparent fluid or tissue, such as for example a blood vessel, the tissue and fluids in the structure 41 may at least partially quench the beam of light. Thus, the beam of light may be at least partially attenuated and reflective illumination may cease or be reduced. When the needle 30 is in place, biological samples may be collected or medical devices or agents may be internally introduced to the target location.

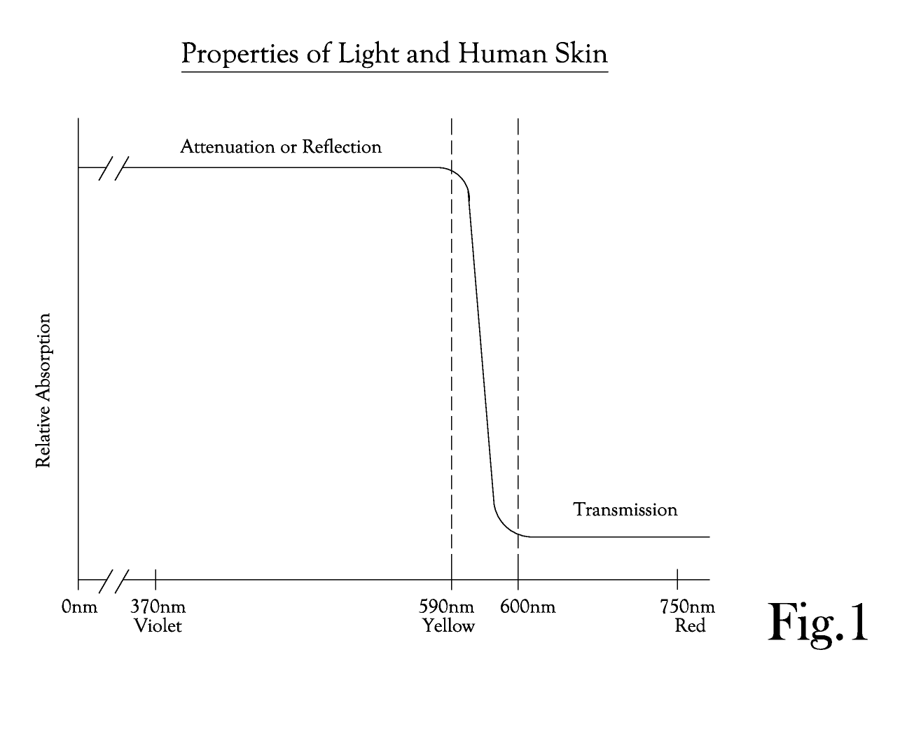

FIG. 1 is an exemplary diagram showing the light attenuation spectrum of a human skin. A waveform, such as light, has several modes of interaction with a physical medium, including transmission, reflection, absorption, and scattering. These may result in attenuation of light energy. Transmission occurs when the waveform travels through the medium without serious disruption, such as when light is transmitted through clear glass. Reflection and scattering occur when the waveform “bounces” off of the medium, while absorption occurs when the waveform is “absorbed” by the medium. The combination of reflection and absorption causes objects to have color when portions of visible light are reflected from their surfaces. Attenuation occurs when energy in the waveform is converted into another form of energy, often thermal energy. If a material attenuates sufficient energy from a waveform, it may melt, sublimate, oxidize, or ablate as a result.

Light comes in many wavelengths that correspond to certain colors and spectral groups, such as red light or ultraviolet light, that react in unique ways with different materials, which in turn interact with the light in any or all of the modes discussed above. The transmission, reflection, and attenuation properties of a particular tissue in the body 40 will be affected by the coloration and chemical composition of the biological material comprising the tissue. Due to differences in skin pigmentation between persons, different wavelengths of light will be effective in different persons, and therefore the specific wavelengths shown in FIG. 1 and discussed herein should be understood to be illustrative. A clinician targeting a structure 41 to access with a vascular access detection device 01, such as blood vessels, will select a portion of the visible spectrum of light that will generally transmit through skin 42, reflect from surrounding tissue in the body 40, and attenuate in the targeted structure 41. For a vascular access detection device 01 adapted for blood vessels, a wavelength between and including the ultraviolet to near infrared spectrums has been found to be effective, because skin 42 transmits light of this wavelength, sub dermal tissue reflects light of this wavelength, and blood and blood vessels attenuate light of this wavelength.

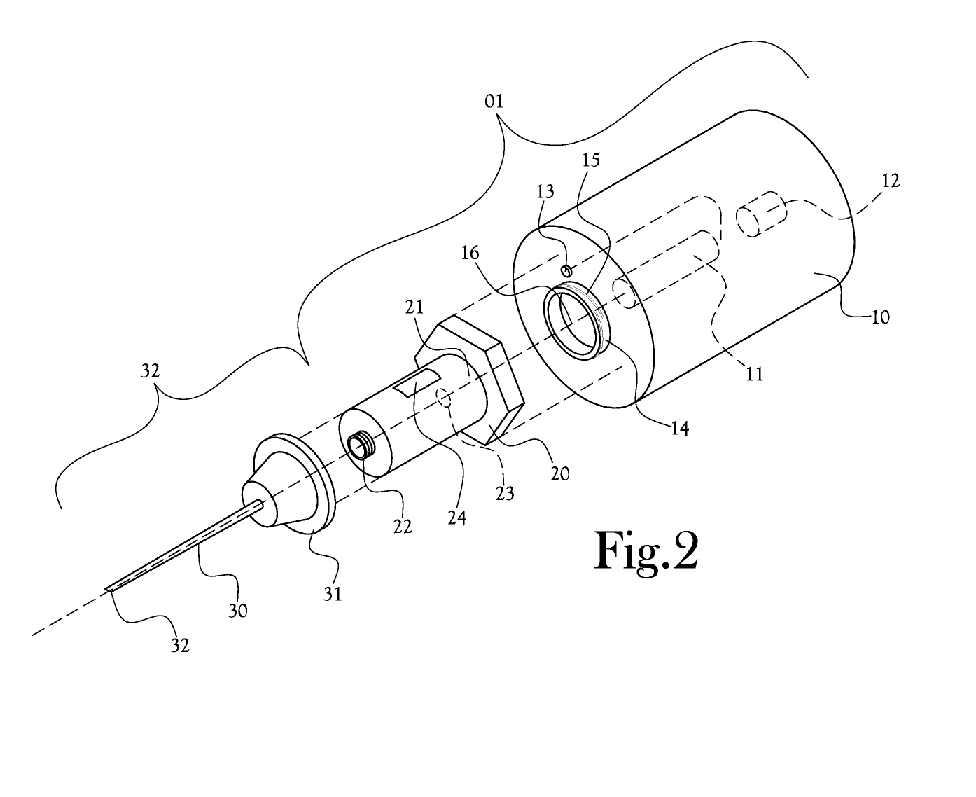

FIG. 2 is an isometric view of one embodiment of the vascular access detection device 01, constructed in accordance with several features of the present general inventive concept, and attached to a needle hub 31. The vascular access detection device 01 is made of two major components, a light source 10 and an interface bridge 20, and is mated with a commercially available needle 30, which is used to pierce the skin 42 and an internal structure 41 during a medical procedure. The light source 10 has an interface connector 15 which houses an aperture 16, which allows a beam of light to exit the light source 10. The interface bridge 20 has a lens, which allows the beam of light to pass through the interface bridge 20, but prevents bodily fluids from passing through the interface bridge 20, an internal connector 21, used for operatively engaging the interface connector 15 of the light source 10, and external connector 22, used for engaging the needle 30. The needle 30 has a hollow shaft and a sharp tip 32 used for gaining access to a pierced structure 41 and a needle hub 31, used for engaging a medical device, such as a syringe or the vascular access detection device 01 at the external connector 22 of the interface bridge 20. Although a needle 30 and needle hub 31 are shown in FIG. 2, the term “needle” is to be understood to encompass all devices, such as trocars and cannulae, used to pierce a patient's skin 42 and deliver medical devices, fluids, and therapeutic agents or draw samples from a patient, and the terms “hub” or “needle hub” are to be understood to refer to the associated interfaces. Both elements, the light source 10 and interface bridge 20, as well as and needle 30, are coaxially aligned along the needle's 30 longitudinal axis so that the beam of light produced by the light source 10 can travel through the hollow shaft of the needle 30 and exit at the tip 32.

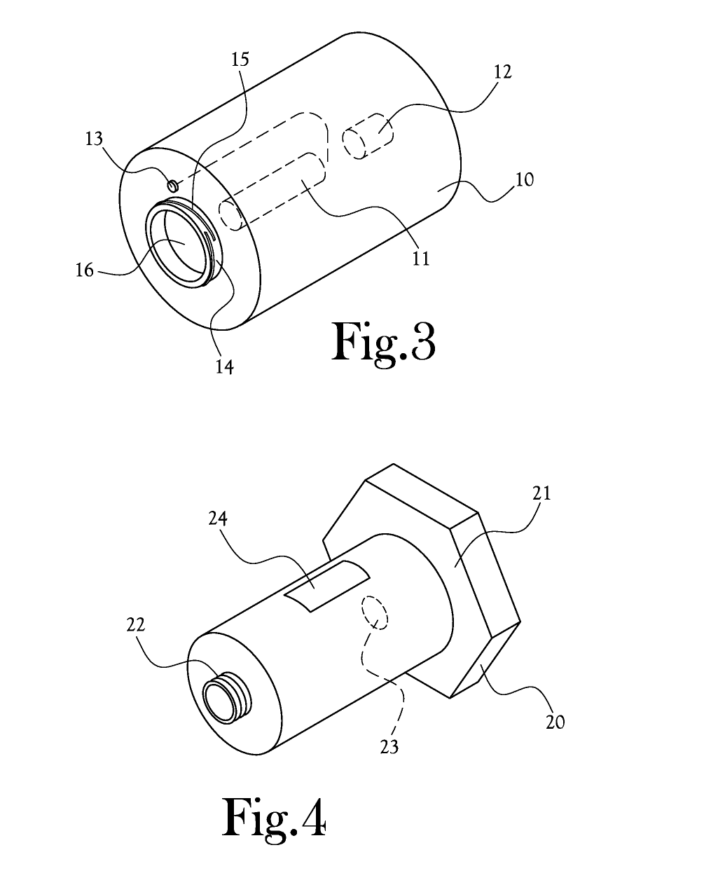

FIG. 3 is an isometric view of one embodiment of the light source 10. The light source 10 is used to generate a beam of light that is directed through a needle 30 and into a patient's body 40, by which a clinician can determine whether the tip 32 of the needle 30 is properly positioned. In the embodiment shown in FIG. 3, the light source 10 uses a laser, which produces coherent light, as its light generator 11, which offers the advantage of requiring less focusing than non-coherent light, but focused non-coherent light will also allow clinicians to ascertain the location of the needle tip 32 within a patient. The light generator 11 is adapted to produce a beam of light that is nonablative; the beam of light is not powerful enough to burn, melt, or vaporize material or tissue that attenuates the beam of light. A nonablative beam of light is adapted for exploratory purposes, not therapeutic uses, which offers the benefit of lower power consumption. The light source 10 illustrated in FIG. 3 is powered by an internal power source 12, but external power sources 12 also are appropriate. The light generator 11 produces a beam of light that exits the light source 10 through an aperture 16. FIG. 3 shows one embodiment of an aperture 16 as a through-hole in the light source 10, but color filters, focusing lenses, and transparent protective caps are used as apertures 16 in other embodiments of the present invention. The term “aperture” is to be understood to include any portion of the light source 10 through which light can pass. In some embodiments, apertures 16 can be designed to filter the beams of light produced by the light generators 11 to exit the apertures 16 at different subsets of the visible spectrum than they are generated at, so that light generated outside a desired wavelength for example, outside a wavelength corresponding to at or between the ultraviolet and near infrared spectrums, will be limited from exiting the apertures 16. In FIG. 3, the aperture 16 is illustrated as part of the interface connector 15 that operatively engages the interface bridge 20 as a male, locking connector, but other connector types, such as snap-on clasps or sockets, have also been successful in engaging the interface bridge 20. The interface connector 15 in some embodiments is sized or shaped to create an interface interlock 14, which prevents the light source 10 from operatively engaging needles 30 or needle hubs 31 and requires the use of the interface bridge 20. The illustrated embodiment in FIG. 3 also includes an optional beam interlock 13, which prevents the light source 10 from generating the beam of light unless the interface bridge 20 operatively engages the light source 10. The beam interlock 13 is illustrated as an electro-mechanical switch in FIG. 3, but other interlock devices and methods, such as requiring a conductive portion of the interface bridge 20 to complete the circuit powering the light source 10, have also been shown to prevent the light source 10 from activating without the interface bridge 20 in place.

FIG. 4 is an isometric view of one embodiment of the interface bridge 20. The interface bridge 20 is designed to be a light-permeable, sterile shield between a patient, into whom the needle 30 is inserted, and the light source 10. Disposable needles come in a wide array of commercially available gauges and connection types, and various embodiments of the interface bridge 20 are sized and designed to operatively engage these various needles. The illustrated embodiment of an interface bridge 20 in FIG. 4 uses internal connectors 21 and external connectors 22 that are illustrated as a locking connector and threads respectively to secure both the needle 30 and light source 10, but other fastener types, such as snap-on clasps or sockets, have also been successful in ensuring proper engagement of the light source 10 and the needle 30. The interface bridge 20, by engaging the light source 10 and the needle 30, aligns a pathway for the beam of light to flow through. To allow light to pass outwardly into the needle 30, while prohibiting fluids from passing to the light source 10 inwardly from the needle 30, the illustrated embodiment of the interface bridge 20 includes a lens 23 made of a substantially transparent material. The lens 23 provides focusing or aiming capabilities in some embodiments, but in the embodiment shown in FIG. 4, the lens 23 is flat and provides no focusing or aiming capabilities to the beam of light. The embodiment shown in FIG. 4 also includes a window 24 located downstream of where the beam of light passes through the lens 23. The window 24 allows an operator to visually verify that the light source 10 is generating the beam of light, which might not otherwise be evident when the needle 30 is properly inserted into a blood vessel or other internal target. The window 24 may be located downstream or upstream of the lens 23 and may be an inset in the interface bridge 20, a hole, or be achieved through an interface bridge 20 substantially made of a material that allows the passage of light.

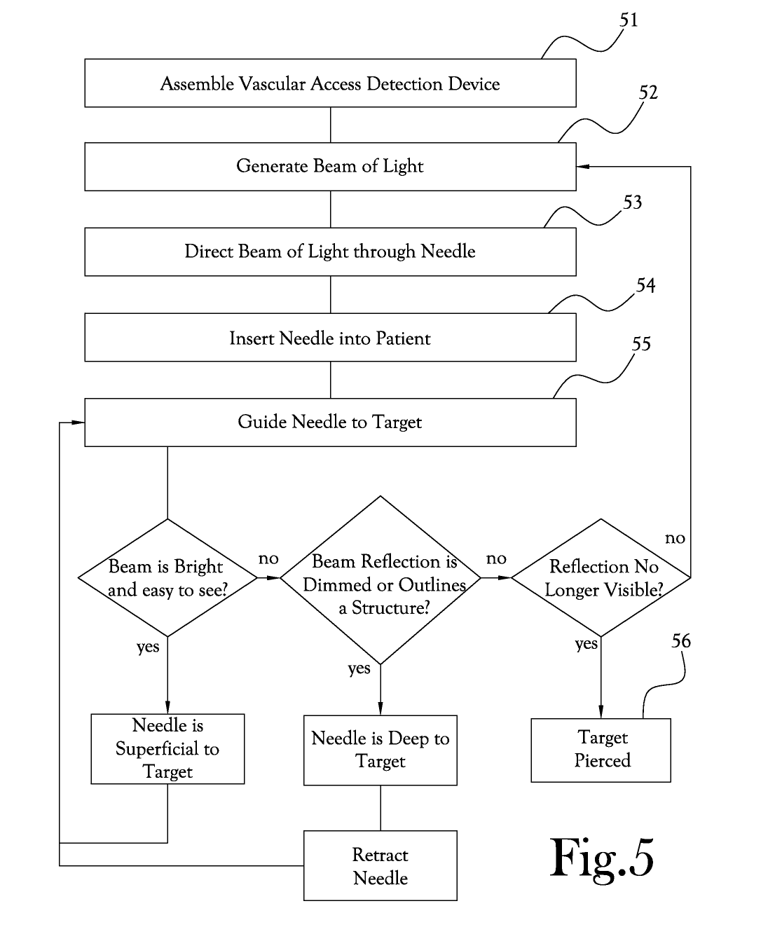

FIG. 5 is a flowchart of one embodiment of the present invention, demonstrating how a clinician can detect vascular access. In one embodiment of the present general inventive concept, the vascular access detection device 01 is assembled and connected 51 to the needle hub 31. The beam of light is generated 52 and it is ensured 53 that the beam of light is directed through the needle 30. The needle 30 is then inserted 54 into the patient, at which time the beam of light may be reflected and scattered off of internal tissue and though the patient's skin 42. The clinician may use the reflected beam of light to reposition and guide 55 the needle 30 based on the intensity level of the reflected beam of light. When the tip 32 of the needle 30 is shallow under the skin 42, the reflection may be bright and easy to see, and the intensity may be dim as the tip of the tip 32 penetrates deeper. When the tip 32 passes under a structure 41, such as a blood vessel, the reflection from the beam of light may be occluded; the structure 41 will block the reflection, but dim reflections may appear around the edges of the structure 41 and the tip 32 should be removed to reattempt piercing the structure 41. The needle may then enter 56 the blood vessel, which may at least partially quench the beam of light. In one embodiment, reflection is completely cut off as the wall of the blood vessel and blood entering the tip 32 attenuate the beam of light. Once the needle 30 is properly inserted into the blood vessel, the clinician can disconnect the needle hub 31 from the vascular access detection device 01 and perform subsequent medical procedures with the inserted needle 30. While particular emphasis is given in FIG. 5 to detecting access to a blood vessel, a skilled clinician will recognize that the same steps can be used to ensure access to a number of internal structures 41 in addition to blood vessels including, but not limited to: cysts, abscesses, joints, bursae, tumors, organs, and glands.

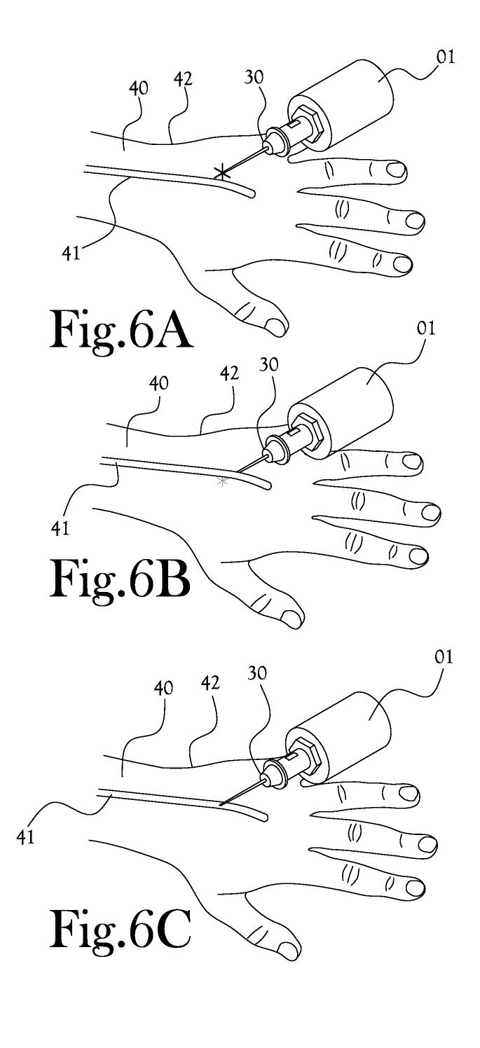

FIGS. 6A-6C are a series of diagrams of reflective patterns for interpreting where the tip 31 of a needle 30 is when using the vascular access detection device 01. Once the tip 31 has penetrated the skin 42, the beam of light may be reflected by the surrounding tissue in the body 40 back through the skin 42, as seen in FIG. 6A. When the tip 31 is shallow in the body 40 and superficial to any structures 41, for example a blood vessel, cyst, abscess, joint, bursa, tumor, organ or gland, the reflection from the beam of light may be bright and easy to see. When the tip 31 is deep to a structure 41, i.e., when the structure 41 lies between the tip 31 and the skin 42, the reflection from the beam of light may dim and be visible on the edges of the structure 41, as shown in FIG. 6B. Once the tip 31 enters a structure 41, such as a blood vessel, cyst, abscess, joint, bursa, tumor, organ or gland, the beam of light may be quenched; the tissue of the structure 41, or fluid within the structure 41, may attenuate the beam of light so that its reflection is no longer visible through the skin 42, as shown in FIG. 6C.

From the foregoing, it will be recognized that a vascular access detection device 01 and method are disclosed which allow a clinician to detect where the tip 32 of a needle 30 is within a patient's body 40. While the present invention has been illustrated by description of several embodiments, which have been described in detail, it is not the intention of the applicant to restrict or in any way limit the scope of the appended claims to such detail. Additional modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

What is claimed is:

1. A vascular access detection device for ensuring the proper insertion of a needle with a hollow shaft held in a needle hub, comprising:
a light source that generates a beam of light, wherein said light source has a distal end, said distal end defining an aperture through which said beam of light passes; and
an interface bridge that protects said light source from contamination comprising a lens that allows light to pass through said interface bridge, wherein said interface bridge is adapted to operatively engage said distal end of said light source and operatively engage the needle hub, wherein said interface bridge aligns said light source and the needle along the needle's longitudinal axis so that said beam of light will pass though the hollow shaft of the needle so as to be detectable.

2. The vascular access detection device of claim 1, wherein said light source further comprises an engagement interlock adapted to prevent said light source from operatively engaging needle hubs.

3. The vascular access detection device of claim 1, wherein said lens focuses said beam of light.

4. The vascular access detection device of claim 3, wherein said interface bridge further comprises a window, said window being adapted to allow an operator to determine whether said beam of light is being generated by said light generator.

5. The vascular access detection device of claim 1, wherein said beam of light is coherent.

6. The vascular access detection device of claim 5, wherein said light source is equipped with a beam interlock, which prevents said beam of light from being generated unless said interface bridge is operatively engaged with said distal end of said light source.

7. The vascular access detection device of claim 5, wherein said beam of light is nonablative.

8. The vascular access detection device of claim 7, wherein said beam of light is of a wavelength in a range corresponding to ultraviolet to near infrared light, whereby blood vessels and blood will attenuate said beam of light so as to signal a user of the vascular access detection device that the needle has entered a targeted structure.

9. A method for detecting vascular access, said method comprising:
a.) aligning a light source, with a needle, said needle further comprising a hollow body and a tip;
b.) generating a beam of light with said light source;
c.) directing said beam of light from said light source through said needle, said beam of light passing through said hollow body to exit at said tip;
d.) inserting said needle into a patient's body;
e.) reflecting and diffusing said beam of light off of tissue in the patient's body so as to be detectable outside of the patient's body; and
f.) quenching said beam of light by piercing a target in the patient's body with said tip.

10. The method for detecting vascular access of claim 9, wherein said beam of light is coherent.

11. The method for detecting vascular access of claim 10, wherein said beam of light is nonablative.

12. The method for detecting vascular access of claim 10, wherein said beam of light is of a wavelength in a range corresponding to ultraviolet to near infrared light.

13. The method for detecting vascular access of claim 9 wherein guiding said needle into the target entails gauging changes in said beam of light's intensity to ascertain where said tip is relative to the target.

14. A method to determine the location of a needle's tip under a patient's skin and within a patient's body, said method comprising:
a.) shining a beam of light through the needle, into the patient's body to reflect through the patient's skin;
b.) guiding the needle to a target inside of the patient's body; and
c.) moving the needle until the target inside of the patient's body has been pierced by the needle and the beam of light is attenuated by the target.

15. The method of claim 14, wherein the beam of light is coherent.

16. The method of claim 15, wherein the beam of light is nonablative.

17. The method of claim 16, wherein the beam of light is of a wavelength in a range corresponding to ultraviolet to near infrared light.

18. The method of claim 14, wherein the target is a blood vessel.
